(19) United States
(12) Patent Application Publication
(10) Pub. No.: US 2003/0172394 A1
(43) Pub. Date: Sep. 11, 2003

(54) Selection and cloning methods

(76) Inventors: George L Holzer, Boise, ID (US); Kathleen Holzer, Boise, ID (US)

Correspondence Address: KLARQUIST SPARKMAN, LLP, 121 SW SALMON STREET, SUITE 1600, PORTLAND, OR, 97204, US

(21) Appl. No.: 10/343,578

(22) PCT Filed: Aug. 1, 2001
(86) PCT No.: PCT/US01/41561

(51) Int. Cl. A01K 67/027; C12Q 1/68

(52) U.S. Cl. 800/21; 435/6; 800/15

(57) ABSTRACT

Selection and cloning methods are disclosed that are effective for the selective multiplication of desired animals, for instance livestock animals. These methods can be used to expand populations of animals, and are particularly useful for duplicating animals selected based on traits that are measured after the animal is deceased. Certain embodiments include techniques useful for selecting a desirable bovine animal (e.g., desirable steer) based at least in part on a measurable carcass trait. Also described are specific cloning techniques that involve repetitive (for instance, two) cycles of cloning, such as nuclear transfer cloning. In specific embodiments, a two-step cloning system is described, in which the nuclear donor of the first cloning cycle is an adult fibroblast cell and the nuclear donor of the second cloning cycle is a fetal fibroblast harvested from a fetus that arose from the list cloning cycle.

### STATEMENT OF GOVERNMENTAL SUPPORT

Research and/or development of certain aspects of this invention were funded, at least in part, through Federal SBIR grant number 1R43HD38140-01AI, granted through the National Institutes of Health. The government may have certain rights in this invention.

### FIELD

The present disclosure concerns methods for animal improvement and multiplication, such as by selecting (e.g., using post-mortem or post-mortem and ante-mortem traits) specific animals (e.g., livestock animals) for clonal production.

### BACKGROUND

Animal Selection

Animal husbandry has long been used to refine and improve desirable traits of domesticated animals. Selective breeding, coupled with carefully managed feeding and medication regimes, has been the traditional method for improving a herd, for instance a herd of livestock animals such as cattle, pigs, goats, or sheep. Competition in the livestock industry, and consumer demand has required meat producers to develop progressively more advanced methods for selecting animals that bear beneficial traits from amongst individuals in their herds.

Original breeding selection techniques involved nothing more sophisticated than looking at individuals in a herd (for example, a herd of cattle), and choosing the best bull and/or cow to serve as the parents of the next generation. Through early selective breeding efforts arose the many different known breeds (varieties) of cattle.

More recently, cattle breeders and producers have developed a relatively sophisticated system to measure and compare several traits between cattle. These traits are referred to generally as estimated progeny differences (EPDs), and data about these traits can be used to predict characteristics about future progeny of a particular sire.

Currently, progeny testing is used to select a bull possessing improved carcass traits on a particular bull. This selection technique is fairly accurate but requires at least four to five years to generate. Progeny testing, which is a part of the calculation of EPDs, is accurate when the repeatability (accuracy) score climbs to the level of ninety percent and higher. This high repeatability requires the addition of many more progeny in many more herds, and therefore the investment of more time to accumulate that data. EPDs can be calculated on younger sires using a combination of their father's EPD for a trait and their mother's EPD for the trait. Most of the mother's EPD for that trait will be derived from her father since she will likely not produce enough offspring to generate her own (reliable) EPD. Progeny-based EPDs of young sires will be of very low repeatability, usually fifteen to thirty percent.

One of the problems of using progeny testing and EPDs as a selection technique to determine the next young sire to use in a breeding program is that there is considerable individual variation. For example, full siblings will have the same EPD for a specific trait (a combination of their parent's EPDs), and yet individually they may perform very differently. This is due to the random assortment of genetic elements from both parents during meiosis.

In addition, post-mortem traits (including carcass EPDs) can only reliably be measurable after an animal has been slaughtered (or has otherwise died). In addition, characteristics are often measured on animals that are sterile (such as steers). For these reasons, it is impossible to use a measured high-scoring animal for breeding stock.

Currently, research efforts focus on identifying individual genes that can be linked to specific livestock traits, such as market traits. Efforts are underway to sequence the genome of, for instance, cattle, and to correlate specific genes or alleles or other markers to specific traits in order to provide better ways to select breeding stock. Though in the future such linkage analysis may provide breeders with readily selectable traits, at the moment this potential has not been realized.

Mammalian Cloning

The birth of the sheep Dolly in 1996 opened the possibility that adult cells could be reprogrammed to act like fertilized embryos and progress, when transferred to a recipient, to the birth of an exact copy of the adult (Wilmut et al., Nature 385:810-813, 1997). Ashworth, et al. (Nature, 394:329-331, 1998) confirmed the authenticity of Dolly's parentage. For some time after the original report on Dolly, numerous laboratories were unable to repeat the experiment. However, the situation has changed recently. Cibelli et al. (Science 280:1256-1258, 1998) have reported the birth of several calves that resulted from the cloning of fetal fibroblast cells. Those fibroblasts carried a “marker” transgene, which conferred resistance to neomycin. The eventual and stated goal of this research is the production of transgenic animals.

Kato et al. (Science 282:2095-2098, 1998) produced eight calves by cloning cumulus cells and oviduct cells. Wells, et al. (Biol. Reprod. 57:385-393, 1997) produced lambs through cloning of an established cell line using in vivo- and in vitro-produced cytoplasts. A live calf has been cloned from cumulus cells of a 13-year old cow (Wells et al., Biol. Reprod. 60:996-1005, 1999).

Vignon, et al. (Comptes Rendus de I Academie des Sciences Serie III-Sciences de la Vie-Life Sciences. 321:735-745, 1998) reported two calves produced by nuclear transfer using muscle cells as genetic donors. This group also reported four bovine pregnancies in late gestation. Of these, one originated from a juvenile female skin cell line and another originated from transgenic fetal skin cells. Zakhartchenko, et al. (Mol. Reprod. Dev. 54:264-272, 1999) produced only a single calf from an adult mammary gland cell and one calf from an adult skin fibroblast. Using goats, Baguisi, et al. (Nat. Biotech. 17:456-461, 1999) produced three kids from fetal somatic cells removed from a transgenic 40-day fetus, which was the product of a mating between a normal female goat and a transgenic male goat. In one of the experimental groups, the couplet was made with an enucleated telophase II oocyte and simultaneously reactivated to induce genome reactivation. Zakhartchenko, et al. (J. Reprod. Fertil. 115:325-331, 1999) also cloned fetal bovine fibroblasts and then recloned using cells from the resulting morulae. The proportion of couplets developing to blastocysts was significantly improved by the recloning procedure.

Another group has reported that a calf had been born from the cloning of skin fibroblast cells (Yang, Transgenic Animal Res. Conf., Tahoe City, Calif., Aug. 14-19, 1999, oral presentation).

In most published reports, the actual conception rate was low and the number of recipients was very low. However, Wells, et al. (Biol. Reprod. 60:996-1005, 1999) transferred 100 cloned bovine granulosa cells to recipients. Quiescent cultured adult granulosa cells were fused with metaphase II cytoplasts using a “fusion before activation” procedure. The rate of blastocyst formation was 27.5% (+/−2.5%), similar to that reported previously (Zakhartchenko et al., Mol. Reprod. Dev. 54:264-272, 1999). After transfer, the 100 recipients produced an initial pregnancy rate of 45%, but only ten calves were born (Wells, et al., Biol. Reprod. 60:996-1005, 1999). This is the largest study reported and confirms the consistent calving rate of approximately 10%.

Cloning of adult cells in cattle has been plagued by low conception rates, high fetal loss rates, and marginal calf survival. From conventional embryo transfer, to frozen embryo transfer, to in vitro produced embryos, to embryos cloned from embryonic cells, and finally embryos cloned from adult somatic cells, conception rate drops, and fetal loss and neonatal calf loss rises. Fetal loss in reported cloning work is often associated with an abnormal allantois and abnormally formed placentomes. This defect suggests that there is inadequate coordination between fetus and mother, rather than a fundamental defect in the cloned fetal tissue.

These major problems with adult cell nuclear transfer (NT) in cattle result in very few of the established pregnancies being maintained beyond sixty days of gestation. In spite of much research, clonal calving rates remain around ten percent (as discussed above). Fetal loss rates and neonatal loss rates are still quite high using the one-step approach. There is a need for techniques that will increase the efficiency of survival of clonal livestock.

The ability to generate viable offspring repeatably from adult cell lines has tremendous agricultural potential. The ability to select cows for premium milk production, steers for carcass production characteristics, and to generate seed stock from these animals, would facilitate genetic improvement greatly and be of great benefit to the economy as a whole, and more particularly agriculture.

### SUMMARY

The inventors have developed a system for selecting and cloning animals with desirable traits, in particular traits that are, or at least typically have been, measured post-mortem. Ante-mortem traits can be incorporated into this selection process, for instance as a pre-selection tool. Using the herein-described system and methods, animals of compromised reproductive capability can be recreated as intact, fertile seedstock, for instance if they possess superior and/or desired traits. This disclosure enables accelerated genetic improvement in animals, leading to more efficient and higher quality animals and improvements in production economics. The quality of meat (such as beef) produced also can be improved using the disclosed methods by selecting for certain characteristics, such as leaner carcasses and increased meat tenderness, that can be measured in an animal after slaughter. The selection and cloning methods of the disclosure find equal application in many different animals, including the several livestock species (such as cattle, pigs, goats, sheep, fowl, and so forth). Methods of the disclosure enable clonal continuation of aged or infirm bulls, as well as multiplication of semen production by clonal production of valuable sires. The described methods can also be used to select for and multiply outstanding dairy cattle, including dairy sires. These techniques can be used to multiply transgenic cows, for instance, cattle producing a therapeutically valuable human protein. In addition, described methods are applicable to the preservation of endangered species.

As a selection tool, an animal's individual performance on a particular trait is a very good indicator of his genetic merit, provided that the specified trait is highly heritable. Most carcass traits are highly heritable. Therefore, selecting the best performer from a large group of animals is believed to be more effective than selecting the animal with the highest progeny-based EPD for that trait. The selection intensity is thereby much greater since the performance bonus of the individual will be much higher than can be found in EPD rankings of even the best bulls available. In addition, using individual performance will allow the next sire to be selected as soon as he is old enough to be fertile (one to three years depending on the reproductive method selected), compared with six to seven years needed to produce a highly repeatable EPD on a variety of sires.

This disclosure provides methods for selecting an animal (or more than one animal) to be cloned, where the animal is selected at least in part based on a characteristic measured after the animal is reproductively impaired. Such impairment can be, for instance, through intentional sterilization (neutering or spaying), disease or accidental sterilization, or death of the animal. In order to clone an animal based on a post-mortem characteristic, a cell sample (from any part of the animal) is taken from the animal, either before or after the animal dies. In certain embodiments, this cell sample is preserved, and it may be cultured in vitro to produce a cell culture. Either cell samples or resultant cell cultures can, for instance, be frozen for preservation.

Animals selected using methods of the disclosure can be cloned. Methods for such cloning, as well as the combination of selection and cloning of animals, are provided.

In certain embodiments, the measurement of more than one characteristic is obtained and considered in selecting the animal to be cloned. For instance, ante-mortem traits can be considered, and can in some instances be used to pre-screen a group of animals in order to assist in selecting an animal to be cloned.

Various characteristics can be used as factors considered in selecting an animal to be cloned. The disclosure encompasses the use of any characteristics, including for instance expected progeny differences (EPDs), governmental quality grades (such as marbling score or meat quality), individual daily gain, and so forth.

Methods of this disclosure can be used to select any type of animal to be cloned, including for instance cattle (and other ruminants), pigs, horses, goats, sheep, chickens, turkeys, mice, rats, monkeys, cats, dogs, reptiles, or captive wild animals.

In particular embodiments, the animal selected is a steer selected from a group of cattle. After such selection, the steer is in some embodiments cloned to produce a genetically essentially identical bull. This bull can further be used to produce semen, which can be collected and distributed for instance for use in modern cattle breeding methods. These processes are also encompassed in the disclosure.

In other embodiments, the disclosure provides a selection process for a female animal, such as a reproductively compromised female bovine. Also provided are methods for selecting and then cloning such a female, to produce an essentially identical female animal, such as a female bovine. Reproductive cells (including eggs, embryos, or fetuses, or cells from such) can be collected from a clonal female animal produced using these methods.

Further embodiments are methods of cloning an animal, where a group of individual animals is assembled, and a cell sample is take from at least one (but usually more than one) of the animals. This sample is preserved, either for long- or short-term storage, and may optionally be converted into an it vitro cell culture. The measurements of one or more traits are obtained for animals of the group, including at least one post-mortem characteristic. These measurements are used to select at least one animal to be cloned, and that animal is then cloned using cells from the cell sample (or the preserved cell sample) corresponding to that animal. In specific embodiments, both post- and ante-mortem traits are used as criteria for selecting an animal, which is then cloned.

The disclosure provides for cloning of animals (e.g., bovine animals) using, for instance, quiescent cell nuclear transfer, proliferating cell nuclear transfer, two-step nuclear transfer cloning, or gonadal cell cloning. For instance, cloning an animal, as provided, can include transferring nuclear material of a preserved cell to a first enucleated oocyte to generate a first couplet, followed by culturing the first couplet in vitro and/or in vivo for a sufficient length of time (e.g., until the first couplet reaches an approximate relative gestational age of 30 days) and under appropriate conditions to produce a first clone. Tissue from this clone can then be put through a second cycle of nuclear transfer cloning, where nuclear material of a cell of the first clone is transferred to a second enucleated oocyte to generate a second couplet; and a cloned animal is then generated from the second couplet. In certain embodiments, the first clone is a fetus when nuclear material is transferred to generate the second couplet.

Also encompassed are methods of selecting and cloning a bovine animal, including identifying a group of bovine animals, preserving a sample from each bovine animal, where the sample contains a fibroblast cell or other types of tissue (cells), obtaining a measurement of at least one ante-mortem characteristic of the bovine animals, slaughtering the group of bovine animals, obtaining a measurement of at least one post-mortem characteristic of the bovine animals, selecting at least one bovine animal to be cloned, based on at least one ante-mortem and at least one post-mortem measured characteristic, and cloning the selected bovine animal from the preserved fibroblast cell. Cloning the fibroblast cell can include transferring nuclear material of the preserved fibroblast cell to a first enucleated oocyte to generate a first couplet, maturing the first couplet in vitro and/or in vivo for a sufficient length of time and under appropriate conditions to produce a fetus of at least 30 days relative gestational age, aborting the fetus or otherwise acquiring a cell sample from the fetus, transferring nuclear material of a fibroblast cell of the fetus to a second enucleated oocyte to generate a second couplet, and generating a cloned animal from the second couplet.

The disclosure also encompasses animals produced by the selection, cloning, and selection plus cloning methods described herein. In addition, the cells, and in particular the reproductive cells, of such cloned animals (e.g., the sperm of clonal bulls or the ova of clonal cows, or the fertilized products of such cells) are also encompassed.

The foregoing and other features and advantages will become more apparent from the following detailed description of several embodiments, which proceeds with reference to the accompanying drawings.

### BRIEF DESCRIPTION OF THE DRAWINGS

FIG. 1 is a flow chart depicting an overall view of the operation of an embodiment of the animal selection and cloning system of the disclosure. Each of the subsystems shown in this figure is described more fully in subsequent figures, and in the text.

FIG. 2 is a flow chart depicting an embodiment, where the animal to be cloned is selected at least in part based on a post-mortem characteristic.

FIG. 3 is a flow chart depicting a two-step method for cloning animals, for instance animals that have been selected using methods of the disclosure. In the depicted embodiment, the first round of cloning uses adult fibroblast cells as the source of nuclear material. In the second round of cloning, fetal fibroblast cells are used.

FIG. 4 is a flow chart depicting one specific selection protocol encompassed by the disclosure. The illustrated embodiment is described more fully in Example 1.

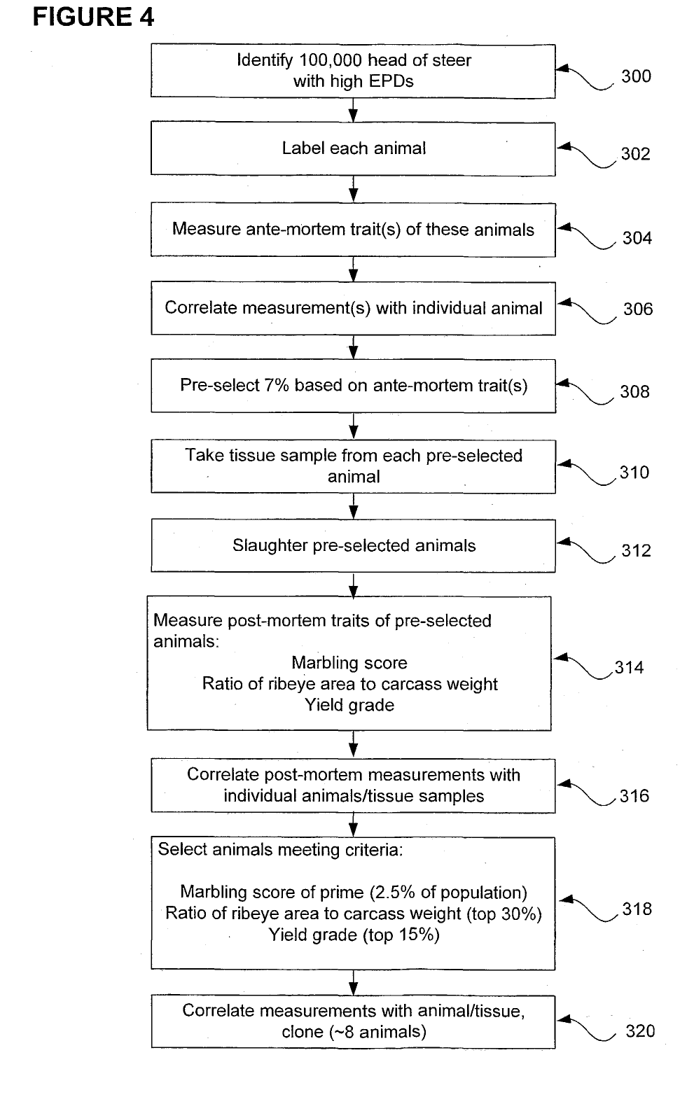

### DETAILED DESCRIPTION

I. Abbreviations and Explanations of Terms

a. Abbreviations

| | |
|---|---|
| EPDs: | estimated progeny differences |
| NT: | nuclear transfer |
| QTL: | quantitative trait locus (loci) |

b. Explanations of Terms

Unless otherwise noted, technical terms are used according to conventional usage. Definitions of common terms in molecular biology may be found in, for example, Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the embodiments, the following explanations of terms are provided. These explanations are not intended to limit the listed terms to a scope narrower than would be known to a person of ordinary skill in the fields of animal (e.g., livestock) selection and cloning.

Animal: Living multi-cellular vertebrate organisms, a category that includes, for example, mammals, reptiles, and birds. Animals can also be divided by type, for instance livestock animals (such as cattle, pigs, horses, goats, sheep, fowl, etc.), laboratory test animals (such as mice, rats, and monkeys), domestic animals (such as household cats and dogs) and captive wild animals (such as may be found in a zoological park). Another category of animals is the ruminants, which are animals that chew their own cud (regurgitate and re-chew previously swallowed food). Goats, sheep, cattle, camels, llamas, elk, deer, and antelope are ruminants.

The phrase “group of animals” refers to any set of two or more animals. A group of animals can be, for instance, as few as two animals or as many as hundreds of thousands. Within any group of animals, all of the animals in a group can be of multiple species (cattle and sheep) or more commonly one species (e.g., all cattle). Additionally, a group can include different varieties or breeds of a single species.

In some embodiments, at least one individual animal within a group is identifiable in some reliable way, such that data taken regarding the animal's characteristics can be correlated with that particular animal. More than one animal within the group, and in some instances all animals of the group, will be individually labeled so they can be correlated with measured data, such as measurements of pre- or post-mortem characteristics. Labeling devices can be anything that will reliably permit correlation, and can include tags attached to the animals (either directly to the animal by way of a piercing, or otherwise such as tied on), brands or dye-stamps (e.g., numbered brands or stamps), implants (for instance implants that include a microchip that is programmable with identifying information), electronic identification tags, etc.

Cattle: General term used to refer to bovine animals, of the genus Bos. Most domesticated cattle are members of the species Bos taurus and B. indicus. A grown male is referred to as a bull; a grown female, a cow; an infant (of either gender), a calf; a female that has not yet given birth, a heifer; and a young, castrated male, a steer. A bullock is a bull in which the testicles have been pushed up against the body of the animal and the scrotum removed, to maintain the testicles at a higher temperature, thereby reducing the violent behavior tendencies of the animal. The term cattle, as used herein, generally refers to all varieties of cattle, as well as crossbred cattle (hybrids between two varieties or two species) and bovine animals of undetermined heritage.

Cell sample: A biological sample that contains at least one cell, optimally a viable cell. Cell samples, as referred to herein, are generally samples taken from any part of an animal, for instance from tissues that include proliferating cells or cells that are capable of proliferating. Cells can be passaged to be used in a quiescent (non-proliferating) stage or a proliferating stages.

Cell samples also include blood samples. Cell samples can be taken from adult animals, from fetal animals, from animal embryos, or from pre-embryonic structures including blastocysts or morulae. Samples that contain more than one cell, or more than one cell type, can also be referred to as “tissue samples” for the purpose of this disclosure.

Specific examples of tissues from which cell samples may be taken include, but are not limited to, gonadal, lung, skin, mammary gland, muscle, bone, glandular, reproductive, lymphatic, kidney, liver, pancreas, spleen, neural, accessory reproductive tissues, hematopoietic tissues, or more generally ectoderm, endoderm or mesoderm. Specific cell types that may form or be found in cell or tissue samples include, but are not limited to, the following: fibroblasts, germ cells, squamous cells, granulosa cells, cumulus cells, and oviduct cells.

Clone/cloned/cloning: Cloning is the creation of a living animal/organism that is genetically essentially identical to the unit or individual from which it was produced. The process of two-step cloning may be used with certain methods, using, for instance adult cells, or adult cells in the first round of cloning followed by fetal cells in the second round of cloning. Other cloning techniques, including simple nuclear transfer, may also be used. In many cloning methods, the clone is not precisely genetically identical to the source organism, for instance due to one or more cytoplasmic genetic elements (e.g., mitochondrial genetic elements) introduced with the recipient cytoplasm. Techniques for mammalian cloning are known, and details can be found for instance in the following patent publications:

U.S. Pat. No. 5,945,577: CLONING USING DONOR NUCLEI FROM PROLIFERATING SOMATIC CELLS;

U.S. Pat. No. 6,011,197: METHOD OF CLONING BOVINES USING REPROGRAMMED NON-EMBRYONIC BOVINE CELLS;

U.S. Pat. No. 6,013,857: TRANSGENIC BOVINES AND MILK FROM TRANSGENIC BOVINES; and

WO 97/07669: QUIESCENT CELL POPULATIONS FOR NUCLEAR TRANSFER.

Depending on the technique, quiescent or proliferating cells can be used in the cloning process. In certain methods, it is beneficial to arrest a proliferating cell (for instance by nutrient deficit or chemical or drug treatment, such as treatment with cytochalasin) during the cloning process.

Couplet: A fused cell, produced through laboratory-assisted means (e.g., nuclear transfer followed by cell fusion, etc.), that contains cytoplasm that is not native to the nucleus. This can be accomplished by transferring the nucleus, or nuclear material, of one cell, or an entire cell, into another (usually enucleated) cell, such as an enucleated oocyte.

Expected progeny differences: An estimate of how future progeny of each individual are expected to perform for the trait specified, in comparison to the national average herd (or a defined average). EPDs for an individual animal can be compared to another individual (a so-called genetic predictor) in order to predict how progeny of the two sires will compare.

An EPD is currently the best estimate of an animal's genetic worth, given the information available for the analysis. Specific EPDs include birth weight, weaning weight, yearling weight, yearling height, mature weight, mature height, milk production, total maternal traits (a combination of milk production and calving ease), postweaning gain, marbling, ribeye area, fat thickness, hot carcass weight, percent retail product, scrotal circumference, mature daughter height, and mature daughter weight. Each EPD is reported in the units of the relevant characteristic—for instance, birth, weaning and yearling weight are reported in mass units, such as pounds, while fat thickness is reported in units of length (depth), such as inches.

The degree of reliability of an EPDs is reflected in the accuracy value. Accuracy values vary from 0 to 1, with values nearer to 1 being more accurate. EPD accuracy reflects the distribution and number of progeny of an animal, the amount of pedigree information available, and the existence of a performance record on the animal. A 0.50 (50%) accuracy indicates that the associated EPD has a 50% chance of being accurate and a 50% chance of being wrong. Sires with large numbers of progeny are more accurately evaluated. In general, an accuracy value of 0.70 or above is considered high.

Governmental quality grade: A system of regulatory standards established by a governing body (or agency thereof) for meat quality. By way of example, in the United States the U.S. Department of Agriculture (USDA) instituted formal grading systems beginning as early as 1923. Codification of early systems has resulted in the U.S. Standards for Grades of Carcass Beef and for Grades of Feeder Cattle (see, Agricultural Marketing Act of 1946 [60 Stat. 1087; 7 U.S.C. 1621-1627], and 7 C.F.R. Part 36). Currently, certain grades assigned to beef by the USDA are (in descending order) Prime, Choice, Good, and Standard.

The USDA has recently proposed updating the governmental quality grading system for feeder cattle (see, 64 FR 51501 and 65 FR 39587) to take into account market changes and genetic improvement. Feeder cattle grades are based on differences in frame size (based on body height and length) and muscle thickness (based on muscle to bone ratio at a given degree of fatness).

Nuclear transfer: For the purposes of this discussion, nuclear transfer (NT) means fusion of nuclear material (e.g., an isolated nucleus or an entire cell) of a donor cell with an enucleated oocyte so that it is reprogrammed to function like a fertilized embryo. This technique is now known, and details can be found for instance in the following publications: Stice et al., Theriogenology 49:129-138, 1998; Solter, Nature 394:315-316, 1998; Wakayama et al., Nature 394, 369-374, 1998; Wells et al., Biol. Reprod. 57:385-393, 1997; Wilmut et al., Nature 385:810-813, 1997. In particular, nuclear transfer has been used, with moderate success, to produce clonal cattle (see, Lanza et al., Science 288:665-669, 2000; Wells et al., Biol. Reprod. 60:996-1005, 1999; Kato et al., Science 282:2095-2098, 1998; Zakhartchenko et al., Mol. Reprod. Dev. 54:264-272, 1999; Zakhartchenko et al., J. Reprod. Fert. 115:325-331, 1999; Kato et al., Science, 282:2095-2098, 1998; Lanza et al., Science, 288:665-669.

Preserving: The general term preserving, or preservation, as used herein, refers to scientifically acceptable methods for maintaining a biological sample (such as a cell sample, or a sample of an its in vitro cell culture) for an extended period of time, such that the sample (or a cell within the sample) is viable at the end of the period. The period of time will vary with the purpose for which the sample is preserved, and the manner of preservation, and may vary from a few hours to weeks or even months. Methods of preserving biological samples, such as cell and tissue samples and in vitro cultures, are various, and include immortalization of cell cultures, cryopreservation (freezing), and/or lyophilization (freeze-drying).

Quantitative trait locus (loci): A genetic indicator, for instance a polymorphism, that is linked to differential quality of an animal carrying the indicator. Mapping markers linked to QTLs identifies regions of the genome that may contain genes involved in the expression of the quantitative trait.

II. Overview of Animal Selection and Cloning

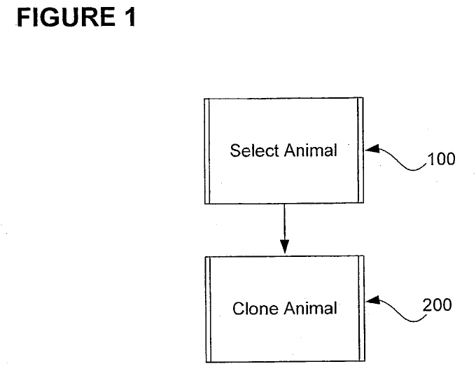

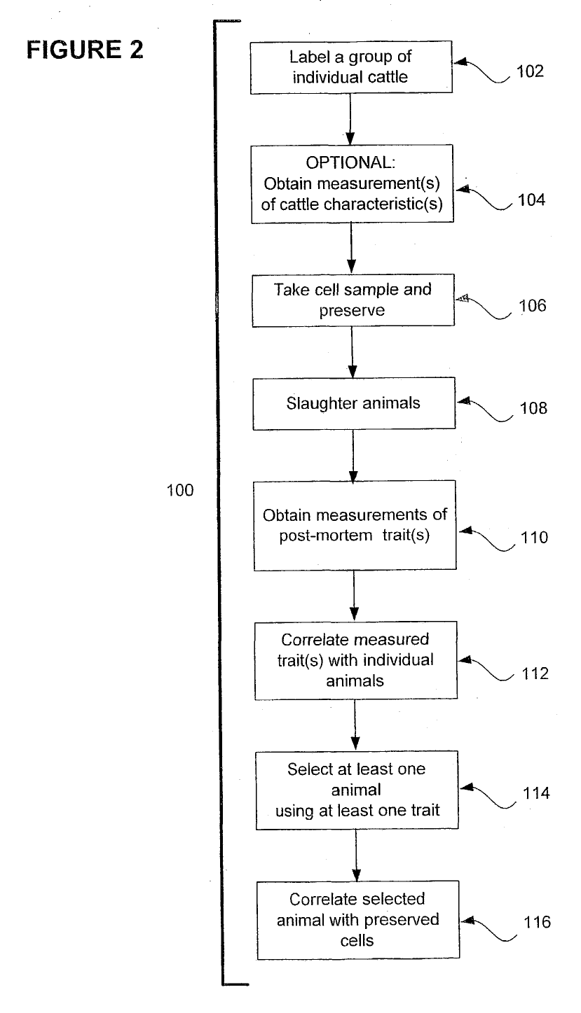

This disclosure provides systems as depicted in overview in FIG. 1 for selecting (100) one or more animals from a group of individual animals, and cloning these animals (200). In very broad overview, selection methods (100) operate are depicted in FIG. 2. A group of animals (illustrated with cattle, but not limited thereto) is assembled and each is identified in a specific and reliable way, i.e., labeled (102). In some embodiments, one or more characteristics of the individual animals can be measured while the animals are alive (104). Cell samples are taken from at least some, but possibly all, of the animals of the group (for instance, from animals that display one or more desirable traits based on live characteristics), and these cell samples are preserved in such a manner that they will provide viable nuclear material for cloning purposes (106). Cell samples can be taken while the animals are alive, or can be taken during slaughter or after slaughter in some embodiments, so long as the cell sample can be used for cloning purposes.

When at least some of the animals of the group are slaughtered (108), or die of natural causes, measurements of one or more post-mortem characteristics are obtained (110). “Obtained” as used herein can mean directly measuring, or getting the data from a third party. The obtained measurements are then correlated to the individual animals and the matching individual cell samples (112). At least one animal is then selected based on at least one post-mortem characteristic (114), and the selected animal is correlated with the appropriate preserved cell sample (116). This selected animal is then re-created through cloning (200) using the preserved cell sample as the source of genetic material. In certain embodiments, other characteristics are used in the selection process including, for instance and without limitation, one or more characteristics that were measured while the animal was alive. Animals selected in this method can be cloned using any method, including known methods as well as those methods specifically described herein. In some embodiments, the selected animals are cloned using a two-step nuclear cloning system.

An exemplary selection system involves the selection of cattle using ante- and post-mortem traits. A group of cattle are identified (for instance, by being assembled) and each animal is labeled in some fashion that enables its future identification. One or more characteristics of the individual cattle are examined and measured, at least one characteristic is measured while the animal is alive, and at least one measurement is taken after the animal is dead (e.g., through slaughter). The inclusion of at least one ante-mortem (live) characteristic permits the optional early “weeding-out” (culling) of certain animals from the group, prior to slaughter. A cell sample is taken from each individual animal. Though the order of taking measurements and taking the cell sample is generally immaterial, in this example the cell sample is taken after measurement of a live characteristic, but before slaughter of the animals. A culling step can be used to reduce the number of animals from which a cell sample is taken.

The animals are slaughtered, and at least one post-mortem characteristic is measured. These data are added to any data collected while the animal was alive, and the data are correlated. The measured traits are then used to select one (or more) animals to clone; the animal can be selected on the basis of particularly good trait measurements, particularly bad trait measurements, or some combination of desirable trait measurements, for instance. The selected animal identification label is then used to match the selected animal to the cell sample taken previously, and preserved. This preserved cell sample is then used to clone the now dead, selected animal.

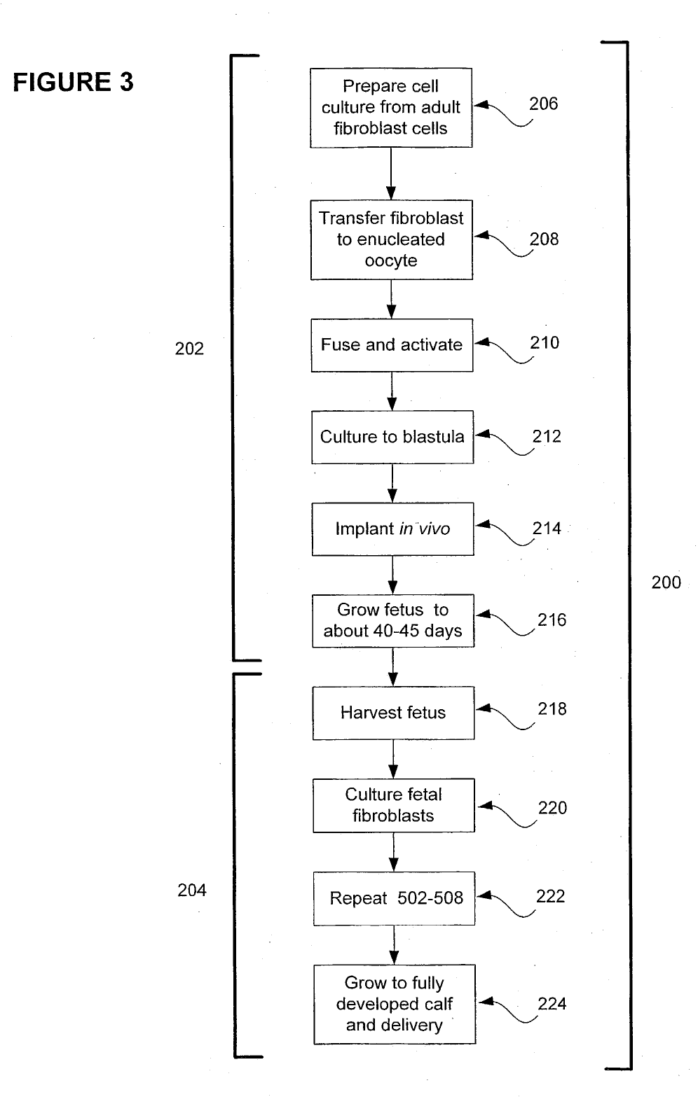

One specific embodiment of a cloning system (200) that can be used is two-step nuclear transfer cloning, illustrated in FIG. 3. The first cloning “step” (202) of two-step cloning involves producing a fetus of 30 or more days, e.g. 40-45 days, using nuclear transfer. In the second cloning “step” (204), a second clone is produced from cells of this first cloned fetus. Adult bovine fibroblasts are used to establish a cell culture (206), which is then used as a nuclear donor for nuclear transfer (208-212). Resultant successful first-round cybrids are transferred to recipient cows, to establish pregnancies (214). The resultant fetuses are termed first-generation (adult cell) clones.

Some of the first-generation adult cell cloned fetuses are sacrificed for the harvesting of fetal fibroblasts (and/or other tissues, such as gonadal cells, or cells from the genital ridge). In certain embodiments, the fetuses are sacrificed at about 30 or more days, for instance at about 45 days (216). Fetal tissue then may be harvested and established (218) in tissue culture (220) and a sample of the cell culture optionally frozen or otherwise preserved. This sample, preserved or fresh, is used to establish second-round cybrids, using nuclear transfer techniques, from which are raised cloned embryos (222). These embryos are transferred to a second set of recipient to cows, thereby producing second-generation clones. These fetuses are permitted to mature utero, to produce live, clonal cattle (224). This process is referred to as two-cycle or two-step cloning.

The two-cycle or two-step cloning technique described here currently is believed to provide much more efficient production of clonal calves compared to other, existing cloning techniques, such as an efficiency of from about 7% to about 20% calving rates, which provides an appreciable increase relative to other known cloning methods (currently yielding no more than about 10% calving). The additional expense in time, resources, and space necessary to carry out the first round of cloning (in order to produce the first clonal fetuses) is minimal (about 5-10% of an overall selection and cloning system), and this additional expense should be recouped by the increase in successful yield using this method.

III. Animals

Embodiments of the selection technique can be applied to any animal for which traits (that can be used as selection criteria) are or can be determined after death. These include, for instance, all manner of livestock or other animals from which meat is harvested, such as cattle, pigs, sheep, rabbits, chickens or other fowl, and so forth. Likewise, the described selection techniques can be used to choose laboratory animals (e.g., mice, rabbits, rats, or monkeys) for clonal expansion, where a post-mortem characteristic is used to identify a desirable phenotype. Conventional cloning techniques may be adapted for use with each of these different animals; the selection techniques as described herein are equally amenable to use with any animal.

In specific examples provided herein, the animals that undergo selection and/or cloning are domestic beef or dairy cattle.

Identification of individual animals, so that measured data can be correlated with the individuals, is important in certain embodiments. In such embodiments, the individual animals are labeled or marked in some manner, and these identifications are usually recorded. One example of a label system that can be used to identify individual animals is an electronic identification (EID) tag system.

Though described herein as a group of “assembled” animals, in certain embodiments the animals that make up a group from which a selection will be made need not be assembled to a single location. Animals can be housed in different facilities, for instance in distantly located feedlots, zoological parks, laboratories, etc., so long as individual animals can be identified and data relating to the individual animals can be correlated. Thus, selection systems are envisioned wherein a network of animal locations and information gathering sites is organized, with one to a few or even several animals at each location. Data that are gathered relating to the ante- and/or post-mortem traits of these separated animals can be compared for the selection of one or more animals to be cloned. However, in certain embodiments, separation of the animals may confound genetic effects by introducing different environmental influences for separated individuals. Such influences may mask or artificially accentuate measured traits, and thus make comparisons between separated animal groups more complicated. It may be beneficial to control for such confounding environmental effects.

IV. Selection for Cloning Purposes

This disclosure provides methods for the selection of individual animals to be subsequently cloned. Such selection can be based on myriad different characteristics of the animals being studied, but has special application in selecting animals based at least in part on one or more traits that are examined or reliably measured after the animal is deceased. The selection methods described herein permit production of animals that cannot reproduce, because, for example, the animal has been made artificially sterile (e.g., through castration), is naturally sterile (e.g., through advanced age or disease), or is dead. These methods can also be used to clone an animal whose reproduction capacity is compromised, for example, by treatment with hormones, other growth promotants, or feeding to an excessively fat condition.

Characteristic(s)

Any (quantitatively or qualitatively) measurable aspect of an animal, whether the aspect is measured in this generation or a future generation, is a characteristic that can be measured for animals in the selection systems. Well-known animal characteristics can be, for instance, examined in the form of estimated progeny differences (EPDs), which are sometimes used to guide breeding decisions in the cattle industry. EPDs, however, are only one of several tools that can be used in order to differentiate between individual animals. Other animal characteristics include indexes and adjusted weights, as well as information on traits such as fertility, structural soundness, muscling, frame size, color, disposition, gait, and so forth. Likewise, the raw or herd ratio data used to calculate EPDs could be used as selection characteristics in methods. DNA linkage markers (such as QTL) or other genetic characteristics can also be used. For instance, the presence or absence of a DNA marker is a genetic characteristic that can be used.

Characteristics of interest can be either live characteristics (measured on a live animal, such as birth weight), or post-mortem characteristics (a subset of which are traditionally referred to as carcass traits, such as ribeye area, yield grade, and marbling). Recently, certain traditionally “carcass” characteristics have been measured on live animals using ultrasound technology (see, for example, U.S. Pat. Nos. 5,836,880, 5,573,002, and 4,913,157).

By way of example only, and in no way meaning to limit the disclosure to selection process involving specific traits, the following is a partial list of animal characteristics/traits that can be measured: birth weight, weaning weight, yearling weight, yearling height, mature weight, mature height, milk production, total maternal traits, individual average daily gain, postweaning gain, marbling, meat taste, meat tenderness, ribeye area, yield grade, hot carcass weight, percent retail product, scrotal circumference, mature daughter height, mature daughter weight, fat type, degree of fat saturation, meat tenderness, meat shelf life, growth rate, feed conversion (efficiency), and age of maturity.

Measurement(s)

One or more characteristics of individual animals are examined and measurements of characteristics obtained. These measurements optionally can be made while the animal is alive, but in most embodiments at least one such measurement is taken after the animal is dead. The time when a characteristic is measured will be in part dependent on what the characteristic is—especially in the matter of post-mortem traits, which as the name implies are traditionally measured when the animal is dead.

The method by which a characteristic is measured also can depend on the characteristic being measured. For instance, where the characteristic being measured is weight (e.g., birth weight, weaning weight, or weight gain after a certain period of time), the animal is weighed using any means. Methods for measuring other characteristics (such as ribeye size or form, or fat thickness) will be known to one of ordinary skill in the relevant art. Where the characteristic is related to a governmental quality standard, the relevant government agency usually provides guidelines for how the measurement is to be taken (e.g., the method for ribbing provided by the USDA, used for evaluating the ribeye area between the 12^{th} and 13^{th} ribs). Measurement of certain characteristics, such as fatty acid saturation level, may require laboratory procedures.

Cell Samples

At least one cell sample is taken from the cattle; the cell sample can be from practically any tissue (e.g., a skin sample, or a muscle biopsy). The order of taking measurements and taking the cell sample is generally immaterial, in that the cell samples can be taken prior to measurement of a live characteristic, or after such measurement. Likewise, cell samples can be taken while the animal is alive or after it is dead, so long as the cell sample is capable of being used as a source for genetic material for cloning the selected animal.

The cell samples are preserved, using any technique that maintains at least a proportion (e.g., at least 10%) of the cells “viable” to the extent that they can be used as the source of genetic material for cloning. Preservation can include the production of an in vitro cell culture from the cell sample. Preservation also may include cryopreservation, either of the starting cell sample (or a portion thereof), or of a cell culture produced from such sample.

By way of example, live tissue samples can be removed from a large number of feedlot steers shortly before slaughter or soon after slaughter, such as within about four hours, and cultured into tissue cultures. These tissue cultures can be preserved, such as by cryopreservation, for use at a later date. The identification designation (e.g., number) of the live animal will be correlated with the identification designation of the tissue sample.

Selection Per Se

Animals are selected based on one or a combination of traits. In certain embodiments, at least one trait that makes up part of the selection criteria is measured after the animal is dead. The specific trait(s) used to select one animal from a group will depend on the end use for which the selected animal is desired. In certain embodiments, selection will be for one or more advantageous traits (e.g., high yield, low fat, good temperament, meat tenderness, etc.), or for a combination of advantageous traits (e.g., low food intake and high meat production, or high meat production coupled with tender meat). In other embodiments, selection can be for an apparently arbitrary trait, such as color or liver size. In still other embodiments, animals (for instance, laboratory or research animals) will be selected for apparently negative traits, such as susceptibility to disease.

In selecting for desirable carcass traits in fat steers, a major problem is the inability to breed the animal possessing those traits. The steer is incapable of reproducing its own genes and, at the time of selection, is dead. With cloning, a steer selected for ideal carcass traits can be reproduced intact (fertile), and his semen collected for selective breeding.

In certain embodiments, carcass traits for each carcass are measured using reliable measuring methods currently available to the meat industry. Many carcass traits may be included in the analysis. Out of the large number of animals that potentially may be included in the study, management can be used to determine which carcass displays the premier score on each of these traits. Live animal measurements and calculated traits involving carcass data combined with live animal measured data also may be utilized to select an animal. In addition, the scores of all the traits for all of the carcasses can be weighted for economic value (importance) and/or heritability. Out of this evaluation, a composite score is developed to determine which carcass displays the combination of traits that are most valuable to the market, the feedlot, the producer, and/or the retail market. Estimated heritability scores for those traits may be used to further weight this scoring process.

III. Cloning Methods

Animals selected using the methods described herein can be cloned using any conventional method, including the cloning techniques described herein, as well as refinements and new cloning techniques.

Cloning of embryos by nuclear transplantation has been developed in several species. Cloning involves the transfer of an adult somatic cell into an enucleated cell, for instance a metaphase II oocyte. This oocyte has the ability to incorporate the transferred nucleus and support development of a new embryo (Prather et al., Biol. Reprod. 41:414-418, 1989; Campbell et al., Nature 380:64-66, 1996; Wilmut et al., Nature 385:810-813, 1997). Morphological indications of this re-programming are the dispersion of nucleoli (Szollosi et al., J. Cell Sci. 91:603-613, 1988) and swelling of the transferred nucleus (Czolowska et al., 1984; Stice and Robl, Biol. Reprod. 39:657-664, 1988; Prather et al., J. Exp. Zool. 225:355-358, 1990; Collas and Robl. Biol. Reprod. 45:455-465, 1991). The most conclusive evidence that the oocyte cytoplasm has the ability to re-program is the birth of offspring from nuclear transplant embryos in several species, including sheep (Smith and Wilmut, Biol. Reprod. 40:1027 1035, 1989; Campbell et al., Nature 380:64-66, 1996; Wells et al., Biol. Reprod. 57:385-393, 1997), cattle (Wells et al., Biol. Reprod. 60:996-1005, 1999; Kato et al., Science 282:2095-2098, 1998; Prather et al., Biol. Reprod. 37:859-866, 1987; Bondioli et al., Theriogenology 33:165-174, 1990), pigs (Prather et al., Biol. Reprod. 41:414-418, 1989) and rabbits (Stice and Robl, Biol. Reprod. 39:657-664, 1988).

One-Step Cloning

The technique of embryonic cell cloning can be used to reproduce animals selected using the methods described herein. However, to use this cloning technique, the cell sample removed from each animal must be taken from the animal while it itself is embryonic. Thus, in order to use embryonic cloning in the selection and cloning methods of the disclosure, the animals used for the selection must themselves have undergone laboratory manipulation at the embryonic stage, for instance being the result of in vitro fertilization, embryo splitting, or another implantation technique.

In embryonic cell cloning, one or more blastomere cells are removed from a young, e.g., six-day-old, embryo. Using conventional techniques, a blastomere is then immediately fused with an oocyte (unfertilized egg cell), which was harvested from an ovarian follicle and enucleated (the native oocyte nuclear material removed). Using the described selection/cloning system, the blastomere(s) are preserved for a period of time, during which traits of the animal from which the blastomere was removed are examined. Blastomeres that were originally harvested from animals that are later selected using the methods described herein are then taken out of preservation and used for fusion to enucleate oocytes.

After fusion of the blastomere to the enucleate oocyte, the NT embryo is cultured for relatively short time (e.g., five days or so) to determine viability (i.e., development to morula stage). This morula is then implanted into the uterus of a surrogate animal. Clonal animals produced using this technique are exact copies of the original embryo from which the blastomere was removed, except for whatever contribution the enucleate oocyte makes.

In 1997, researchers at the Roslin Institute announced the production of the sheep Dolly by cloning her mammary tissue (Wilmut et al., Nature 385:810-813, 1997). Since then several laboratories have reported the production of a fairly small number of calves cloned from adult cells. This process involves the fusing of the nucleus of an adult cell with an oocyte from which all genetic material has been removed (an enucleated oocyte). After short-term in vitro, or in vivo, culture, viable embryos are transferred to surrogate recipient cows for completion of gestation. The initial conception rate for this system has been fairly low and a very large percentage of the pregnancies have been lost before calving.

Cloning can also be performed using the nucleus of an adult cell. In adult cell cloning, an adult somatic cell (i.e. a fibroblast) is fused with an enucleated oocyte. After culture, many of the fused couplets (or cybrids) develop into morulae. When these morulae are transferred to recipient cattle, the reported conception rate is about 30-40%. However, the proportion of the fetuses that persist beyond sixty days gestation has been only about 5-10% (Wells et al., Biol. Reprod. 60:996-1005, 1999).

Two-Step Cloning

Two cycles of cloning can be carried out in order to increase the efficiency of production of cloned calves. This cloning system is referred to herein as “two-step cloning,” “two-cycle cloning,” or “two-step nuclear cloning.”

Two-step cloning involves a first cloning cycle (e.g., by nuclear transfer) using an adult cell, growing the resultant cybrid in vitro and/or in vivo to produce a clonal fetus, then using a fetal cell from the clonal fetus for a second round of cloning (e.g., also by nuclear transfer). This procedure provides more efficient production of calves from adult cells. In one example, a fibroblast from an adult animal is fused with an enucleated oocyte and cultured to about the morula stage. The viable morulae resulting from this procedure are transferred to recipients. Most of these first-cycle pregnancies can be allowed to attempt to reach term, for instance for use as an internal experimental control. After the embryo has developed into a fetus (generally for a sufficient amount of time to display differentiation into tissues and organs), at least one and up to several of these first-cycle fetuses are removed surgically to provide tissue for the production of tissue cultures. By way of example, cattle fetuses can generally be used after they have reached a gestational age of at least 30 days; in specific embodiments, cattle fetuses can be sacrificed at about 45 days gestational age. Any fetal tissue can serve to produce fetal tissue cultures. In representative embodiments, fetal cell cultures are produced from fetal fibroblasts or gonadal cells or cells from the genital ridge. The fetal cell cultures are propagated and samples preserved (e.g., frozen) for future use. In certain embodiments, fetal tissue is used directly for the second round of cloning (without an intervening storage stage, and in some instances without development of an in vitro cell culture).

The fetal cell cultures (e.g., fibroblast cultures) can be used as nuclear donors for the second cloning cycle. In this second cycle (the second “step” of two-step cloning), fetal cultured cells are fused with enucleated oocytes to produce second-generation morulae. These morulae are transferred to recipients and the resulting pregnancies allowed to go to term to produce live progeny. This two-step cloning procedure is expected to result in, for instance, a clonal progeny production rate of 30-40% in cattle, based on conception rates established for embryonic cell cloning. Without meaning to be bound to one theory or explanation, the inventors currently propose that a reprogramming of the genetic clock occurs during early embryonic development and that two cycles of early embryonic development will result in an improved calving rate. The resulting calves are exact copies of the adult animal from which the adult cells were originally removed (except for any influence that may be exerted by cytoplasmic elements introduced during the cloning process).

Adult cells (either proliferating or quiescent) are used as nuclear donors to produce nuclear transfer cloned embryos or fetuses (for instances, fetuses of about 40-45 days). These embryos/fetuses are used to establish cell lines. Cells from the cells lines are then used as nuclear donor cells to produce second generation cloned embryos, which are transferred to recipient animals and carried to term.

As discussed more fully below, the nuclear donor cells in either the first or the second cycle of cloning optionally can be transgenic.

Fibroblasts are proposed as a starting material in certain of the specific embodiments disclosed, since fibroblasts are present in male as well as female specimens. Fibroblasts are readily cultured, but other cell types can be used in the methods described herein.

Pregnancies resulting from the transfer of fetal-origin, second-generation cloned embryos are allowed to mature for the full gestation period and result in the delivery of live calves.

VII. Transgenesis

Development of transgenic animals that produce therapeutic human proteins provides opportunities to reduce the cost of these products by a factor of ten and in some cases as much as one hundred. Many human genetic diseases exist in which infants are born with a defect in protein metabolism. Sometimes the cause is genetic, and sometimes there is an error in fetal development. Many hundreds of millions of dollars are spent each year to extract these proteins from blood supplies and cadavers for administration to these patients. Cows transgenic for these genes can produce many of these proteins in their milk at a fraction of the current cost. Transgenic protein production avoids the risk of disease transmission inherent in products developed from human blood banks and cadavers.

The production of transgenic bovines is known. Techniques for producing transgenic bovines can be found for instance in the following: Cibelli et al., Nat. Biotech. 16:642-646, 1998; Cibelli et al., Science 280:1256-1258, 1998; and Brink et al., Theriogenology 53:139-148, 2000.

Transgenics depends heavily on cloning. If embryonic blastomeres are transfected, cloning is used to produce viable embryos. In addition, recent research indicates that transfecting a bed of tissue culture cells with a transgene and a marker gene may increase the efficiency of the transformation process. The development of efficient adult cell cloning procedures will be essential to the implementation of these recent developments. Once a founder transgenic animal is produced, cloning procedures are used to increase the number of animals available, e.g., for the production of therapeutic protein. The selection and cloning methods of the disclosure can be used effectively with transgenic animals and in the production of such animals.

Adult cell cloning applies directly to the field of transgenic production of therapeutic human proteins by cows. The goal is to insert a gene so that a cow will produce a biologically active human protein in her milk. The health aspects of administering transgenically-produced proteins to human patients will require that the producing cattle be specific-pathogen free, to provide assurance that no pathogens are passed with the transgenic protein (e.g., in milk). To ensure this, production of large numbers of cattle embryos by in vitro fertilization of random oocytes collected at slaughterhouses will be replaced with clonal expansion of guaranteed “clean” animals.

In vitro-fertilized one and two cell eggs or embryos are currently used in the transfection process. The rate of incorporation of the transgene is low using this procedure, and the viability of the embryo is poor. Due to time constraints, expression of the transgene by the embryo cannot be determined before the embryo must be transferred to the recipient. Consequently, embryos expressing the transgene, as well as the large number of embryos not expressing the transgene, must be committed to recipient mothers. The recipient expenses in this situation are therefore huge. The milk producing capability of the resulting transgenic cow is unknown at the outset because she results from the oocyte of an unknown animal, randomly selected at the slaughterhouse.

For these and other reasons, the inventors propose that transgenic animals can be produced through transfection of a large number of cultured fibroblast cells removed from the bull or cow with high milk production traits, for instance one selected from a national herd or conglomerate group of animals. The transgene may incorporate a marker gene (e.g., for a production of a dye or antibiotic tolerance) that can be used to identify those fibroblasts that have incorporated the transgene. The few cells which effectively incorporate and express the transgene are identified and used as donor cells in the adult cell cloning procedure(s) as described herein, to produce a live calf. This process allows scientists to start with fibroblasts or other cell types from an excellent milk producing animal, screen these cells for any hidden viruses or other pathogens to establish that the cells are specific pathogen free, freeze aliquots of cells, and use them in the transfection process.

Specific lines of these fibroblasts, for instance those that show exceptional cloning capability, can be frozen for repeated use. These fibroblasts will then be transfected with the human gene and the marker gene. After several days of culture, the transgenic fibroblasts are isolated and cloned. Using this procedure, all of the resulting viable embryos are transgenic embryos. The best of these transgenic embryos can be selected for transfer to recipients.

Using this procedure, the rate of blastocyst formation will not be critical, since all the blastocysts that form are transgenic. The pregnancy rate and fetal survival rate will not need to be comparable to conventional embryo transfer in cattle. However, at this time it is believed that the described two-step cloning procedure will greatly improve the pregnancy rate and fetal survival rate, and possibly the calf survival rate.

Animals selected for one or more beneficial trait as described herein (for instance, animals selected for optimal meat production or flavor) are prime candidates for the introduction of one or more transgenes. Inserting a transgene into a highly selected individual will produce an animal having the ultimate combination of selectable and engineered genetic traits. Whether starting with the herein-described selection process, or designing a customized selection process to accommodate the successful addition of the transgene, this technique will produce the best candidate cells for transfection. If the trait conferred by the transgene is only observed in the carcass, then the animals produced by the transgenic procedure must be slaughtered to confirm the expression or lack of expression of the transgene.

Clonal Expansion

As transgenesis progresses, transgenic animals, such as cows and other livestock, can be made to produce virtually any protein. These products would include critical metabolic products, antibacterial agents, antiviral agents, anti-cancer agents, hormones, enzymes and cell growth promoters, and inhibitors. Bacterial, yeast, and mammalian cell culture systems suffer from the problem of being unable to complete the modification (protein folding and glycosylation) of these products. These post-translational modifications are essential to maintaining biologic activity in the human patient. The bovine mammary gland accomplishes production of complex proteins particularly well, including proper protein folding and glycosylation.

Currently, the production of one transgenic cow that successfully incorporates a human gene is a long arduous process requiring thousands of attempts. The cost of producing just one transgenic cow has usually been over four hundred thousand dollars. Once one of these cows is produced, the multiplication of this cow becomes very important.

The commercial application of the described cloning techniques for the multiplication of transgenic cows provides real and profitable advantages. When transfecting a one-cell or two-cell fertilized embryo, the transgene may be incorporated into only a portion of the embryonic cells (a phenomenon called mosaicism). Mosaicism is very common, and detrimental. Production of a human protein in the milk of a transgenic cow may be low if only 30% of the lacteal cells contain the transgene.

When adult fibroblast cells are transfected and selected for expression of the transgene, each fibroblast gives rise to a cloned calf in whom 100% of the lacteal cells are transgenic. The resulting cow is capable of producing milk much richer in the desired human protein.

Mosaicism creates a similar problem when breeding a transgenic cow to produce transgenic offspring. One would expect that half of the calves of a transgenic cow would contain the transgene. However, if the transgenic cow is a mosaic, then some of her oocytes will contain the transgene and some will not. Due to mosaicism, much fewer than half of the calves will be transgenic, since only a portion of the primordial oocytes are actually transgenic.

Cloning also may be essential for the production of herds of cattle from which specific genes have been knocked out (negative or minus transgenics). For example, knocking out the prion gene in cattle would render them immune to bovine spongiform encephalitis (see, e.g., U.S. Pat. No. 5,962,669). Since many human medicines contain products derived from cattle, such as collagen, disease-resistant knockout cattle may be a unique source for certified prion-free medical products (Wilmut, Sci. Am., 279:58-63, 1998).

During transgenesis, the transgene is incorporated into a random chromosome of the very early embryo. If this embryo survives to produce a transgenic cow, the single transgene functions as though it were a dominant gene since there is no matching gene on the homologous chromosome. A transgenic female will produce a hypothetical X milligrams of human protein per milliliter of milk. If one then breeds this cow to an unrelated male (because there exists no other animals with the transgene located at that specific site on that chromosome), both transgenic and non-transgenic calves will result. If one then breeds the transgenic female back to one of her transgenic sons, progeny can be produced that are homozygous for the transgene. This second-generation transgenic animal has two copies of the transgene at the same location on the two homologous chromosomes. Females with two homologous transgenic chromosomes produce 2×milligrams of human protein per milliliter of milk.

Due to the 30-month generation interval in cattle, this procedure is extremely time-consuming. Sperm and egg formation likely will suffer some loss of the transgene. Mosaicism will also decrease the number of eligible matings. Cloning adult tissue cells of original transgenic animals, to produce second generation and homozygous transgenic animals, will be more productive than attempting to increase their numbers by backcross breeding. Though it will still be necessary to backcross the transgenic animal once to achieve homozygosity of the transgene, the cloning techniques described herein can be used to accelerate production of offspring, for instance coupled with in vitro fertilization techniques, once the homozygote is achieved.

V. Further Applications

The selection for cloning methods, and subsequent cloning methods, described herein can be used beneficially to accomplish several goals, particularly in animal husbandry, animal preservation, and broad applications of transgenesis.

As described above, and in examples more fully detailed below, the selection and cloning techniques of the disclosure can be used to produce improved livestock animals, including animals selected for one or more carcass traits.

These methods can also be used to increase or replicate the reproductive vigor of livestock sires. Occasionally a sire of major economic importance will cease production of semen prematurely, for instance due to a disease condition. There are some sires of importance worldwide whose health status prevents their use in certain foreign countries. Cloning of adult cells from these selected animals would provide a new source for the continual production of their valuable semen. In addition, the supply of semen from a highly desirable sire could be doubled or tripled through supplemental production by one or two clones.

Vanishing and endangered species, or unusual variants or mutations, could be reproduced using the techniques described herein. Adult somatic cell cloning could preserve a species of animals that are nearly extinct, and help maintain an acceptably diverse gene pool for the preservation of less endangered species. These techniques, with some possible modifications for certain species, may be applicable to every endangered species. However, interspecies embryo transfer may present some problems.

Animals identified using the selection methods described herein provide unique opportunities to test the linkage of genes to economically important or other selected traits. A genomic scan (on the clonal sire and/or his offspring or clonal sibs) can be performed using currently available markers. The resulting data are then used to identify Quantitative Trait Loci (QTL's) specific for particular carcass traits. Because artificial insemination with this semen can provide a large cohort of half-siblings, selected for specific traits, the identification of QTL's for carcass traits would advance at an exponential rate.

A superior bull produced by the described selection/cloning system can be bred to a modest number of females. Genomic scans for known genetic markers on the sire and his offspring can be evaluated, along with his carcass trait measurements (previously recorded from the clonal source “parent” of the selected sire, and only available through the herein described selection/cloning techniques) and the carcass traits of his offspring. The outcome of this unique family study, made possible by the techniques disclosed herein, is more rapid and more reliable elucidation of the predictive value of known QTL for the carcass traits of interest. Similarly, new QTL's may be located and their efficiency for predicting these traits can be developed. Negative genetic markers for undesirable traits may be suggested by these studies. One substantial advantage of using the described selection techniques (to produce the sire in this family study) is that the numbers of females, and their calves, needed to evaluate a marker is far fewer than would be the case if one started with a sire possessing only a high EPD for desirable traits, without sacrificing the statistical significance of the experiment.

Adult cell cloning, in particular the two-step cloning systems described herein, are useful for increasing the numbers of very select individual animals. Beef sires that have been shown to carry a high proportion of marker genes, and produce highly desirable feeder cattle progeny, could be mass-produced and marketed to commercial beef producers. If a desirable gene such as disease resistance is inserted into cattle (to yield a transgenic animal), animals carrying the gene must be multiplied in order to spread this trait throughout the national cattle herd. The tremendous value of dairy cows transgenic for a human therapeutic protein will require that adult cell cloning be employed to produce multiple copies of this animal that will be housed in separate secure facilities.

When developing therapeutic or production drugs for animal agriculture, the two-step cloning procedure can be used to create multiple copies of an experimental animal for assembly of the control and experimental groups. Since these two groups of experimental animals will be essentially “identical twins”, the genetic variation between experimental animals is zero. Consequently, the number of animals in each group can be greatly reduced, without sacrificing statistical reliability. If it is desirable to select for a certain post-mortem observed trait, then the above selection process must be use in conjunction with cloning, to produce the family of identical clones for use in the drug study.

Genes for an identity trait or marker protein or genetic marker can be inserted into the genome of a proprietary line of livestock so that the lineage can be proven. These types of identity markers can be inserted into lines of cattle (or other animals), then used to verify ownership or confirm compliance with a contract. Insertion of a marker gene or trait easily can be accomplished using transgenic methods cited herein or known to one of ordinary skill in the art. For those marker traits that can only be observable post-mortem, or which are more accurately or beneficially observed post-mortem, the described selection/cloning methods are necessary to observe and select for the animal with the desired marker. One example of such a marker that is preferentially measured post mortem is a fat marker that would identify offspring of a selected bull in the packing plant, so that it was possible to determine carcasses for which a premium is paid. This readily identified marker characteristic would allow the packer to pay a premium for the carcass without having to do a chemical analysis on each carcass.

All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1

Selection of Cattle with High Economic Traits

This example provides one selection system for animals, particularly cattle, based on at least one post-mortem trait.

Source of the Cattle:

One hundred thousand steers are purchased from ranches known to produce high quality cattle (300, illustrated in FIG. 5). These ranches purchase bulls and retain stock exhibiting high EPDs for maternal, performance and carcass traits. To aid in identification of these cattle, each can be individually labeled (302), e.g. by eartag.

Management and Care of the Cattle:

In order to mitigate potential environmental differences, the cattle are placed on feed in several feedyards, which incorporate the same heath and nutritional programs. The cattle are given identical growth hormones, vaccines and wormers. The cattle are put on feed under the essentially same protocol and fed essentially identical diets.

The cattle are fed for a term using best management practices to achieve the optimum of cattle performance and carcass quality. Techniques for achieving this are well known. The cattle are all fed in feedyards located in a tight geographic area, to minimize differences in the population due to weather. The wet manure in the pens is managed uniformly to create essentially the same pen conditions across the population.

Pre-Harvest Measurements

The cattle are individually weighed when they are initially processed at the feedyards, and are re-weighed at approximately ninety days prior to slaughter (304), when their terminal implants (of hormones) are administered. Measurements are correlated to individual animals (306). Cattle that represent the top seven percent of the population for daily gain, based on these weights, are pre-selected and identified with eartags (308). This is an alternative to labeling all animals in the original group. The selection process may incorporated other pre-harvest criteria, including but not limited to color selection, animal temperament, ribeye area (as estimated by ultra-sound or other methods), known DNA markers for traits, etc. Cattle judged to be exceptional are identified and returned to their home pen (within the feed yard) with the other cattle. Ante-mortem measurements may be taken at intervals in the feeding program, for instance when the animals would be handled anyway for processing. Coordination of such measurements minimizes incremental labor (and associated costs) and stress to the cattle.

Sort Identified Cattle from their Home Pen Prior to Harvest

The exceptional cattle, identified by eartag, are sorted from their home pen prior to harvest. Sorting cattle prior to harvest increases the selection pressure for live (ante-mortem) traits and known DNA markers, since its enables increasing the size of the population without any incremental cost. Pre-selection based on live traits also reduces the number of tissue samples required and carcasses screened at post harvest.

Take and Correlate Tissue Samples

Tissue samples are taken from each of the pre-selected cattle, either before (310) or after slaughter (312). Samples may be taken in the form of ear punches, other skin punches, lung samples, muscle samples, etc. Each sample is marked so that it can be correlated with the originating animal, for instance by using the same number that is on the eartag of the animal. The identification number also enables cross referencing of pre-harvest and post-mortem measurements to the individual carcass and tissue sample.

Process Tissue Samples

Tissue samples are prepared for transport and short-term storage, and shipped to a laboratory. At the laboratory, tissue cultures are generated from the tissue samples, and portions of the cultures are cryopreserved for future use, including use in cloning the selected animal(s).

Measure Post-Mortem Trait(s)

The carcasses are measured for beef marbling, yield grade, and ribeye area (314). They also may be measured for meat and fat color, fatty acid ratio, retail yield, meat tenderness and taste, and shelf-life. Cell samples taken from at least some of the carcasses are analyzed at pre-harvest or post-mortem; known DNA markers are used to identify maternal, production and meat quality traits. These data are correlated (316) with individual tissue samples.

Final Selection of Tissues to be Cloned

Tissue samples that will serve as the source of material for cloning experiments are selected (318) based on the economic value of the individual source animal, and the heritability of the traits that make that animal valuable. The initial selection process focuses on rate of live weight gain, marbling score, ribeye area and yield grade. The selection process also eliminates carcasses that exhibit below average meat and fat color, fatty acid ratio, and tenderness. Thus, the selection process selects the top carcass(es) in terms of retail yield, ribeye area and yield grade, using measured characteristics that provide an accurate estimation of these trait. These measurements are correlated (320) with tissue samples and the sample(s) selected can be used for cloning.

Using the following criteria, this selection process will screen the top eight bulls out of 100,000 candidates (an Estimated Selection Pressure of 7.875/100.000=0.00787% of population) (See Table 1).

TABLE 1

| Selection | Number of cattle (head) |
|---|---|
| Initial Population | 100,000.0 |
| Pre-Harvest Population (7%) | 7,000.0 |
| Marbling Score of Prime (2.5%) | 175.0 |
| Ratio Ribeye to Carcass (top 30%) | 52.5 |
| Yield Grade 1 or better (15%) | 7.875 |

### Example 2

Two-Step Cloning

This example provides one method for cloning bovines that have been selected for one or more desired trait, using the selection methods described herein.

In order to provide control animal sets for quality and efficiency assessment, multiple treatment groups can be established. The first treatment group consists of 1^{st} generation NT embryos (Fib1), produced from adult cattle fibroblast cell lines. Treatments for the Fib1 NT embryos consist of oocyte collection, establishment of primary cell lines, nuclear transfer procedures, activation of MII oocytes, and transfer to synchronized recipients. Resultant pregnancies are allowed to develop to term, with the exception of a minimal number that are sacrificed for production of the second treatment group.

The second treatment group consists of 2^{nd} generation NT embryos (Fib2). Treatments for the 2^{nd} generation NT embryos include oocyte collection, establishment of fetal cell lines similar to the primary cell lines but derived from Fib1 fetal tissue, nuclear transfer procedures, activation of MII oocytes and transfer to synchronized recipients. A few of the pregnancies are aborted and tissues collected for preparation of fetal tissue cultures. The 2^{nd} generation embryos are produced by sacrificing one or more 1^{st} generation pregnancies (established from NT Fib1 embryos derived from adult cell nuclear donors) at about 40 to 45 days gestation. Fetal tissue is removed, and fetal cell lines are established. These fetal cells are used as the nuclear donor cells to generate NT embryos (Fib2).

Recipient animals receive three to four 1^{st} generation embryos each, to maximize the potential for pregnancy. Recipients for 2^{nd} generation embryos receive twins. Pregnancies are determined by ultrasound at 24 days gestation and 2^{nd} generation pregnancies monitored on a weekly basis through about 120 days gestation. Embryos can be transferred surgically so as to maximize the conception rate.

The criteria to be used for determining success include the production of pregnancies beyond ninety days of gestation. There is ample evidence that most NT pregnancies are lost between day 45 and day 70 of gestation (Cibelli et al., Science 280:1256-1258, 1998; Wells et al., Biol. Reprod. 60:996-1005, 1999; and Zakhartchenko et al., J. Reprod. Fertil. 115:325-331, 1999). However, the nearer to calving the clones are carried, the more successful the procedure is. Post mortem examinations will be performed on any aborted fetuses and non-surviving calves for determination of cause of death and possible tissue analysis.

Establishment of Primary Cell Lines

Adult fibroblast cell lines are established from dermal/muscle biopsies. Feedlot steers are monitored for several important economic traits (rate of weight gain, feed efficiency, etc.), sacrificed, and carcass traits evaluated. Tissue samples are removed immediately after kill, placed in PBS containing 10× antibiotics, and immediately transported to the laboratory. Initial explants are cultured in TCM-199 containing 10% FBS and 3× antibiotics. Samples from one to three selected carcasses (graded as prime, yield grade 1 or 2, maximum ribeye area, and adequate marbling) are converted to tissue cultures. Once cells begin to attach and establish, the concentration of antibiotics is reduced to 1×, and aliquots are frozen in liquid nitrogen. Prior to use in NT, these preserved cells are thawed, then “starved” to induce G₀ by culturing in 0.5% FBS for 7-10 days.

Nuclear Transfer Procedures

A donor fibroblast cell line, established from a biopsied carcass tissue (e.g., fibroblasts), is cultured in TCM-199 supplemented with 10% FBS. The donor cells for nuclear transfer are synchronized in G₀ phase of the cell cycle by culturing in TCM-199 plus 0.5% FBS for 7-10 days. Cumulus-free MII oocytes are incubated for 10 minutes with 10 μg/ml of Hoechst 33342 (unless indicated otherwise), transferred to 100 μl of HECM/Hepes manipulation medium containing 7.5 μg/ml cytochalasin B and incubated for 10-15 minutes before enucleation. The first polar body and metaphase plate of an oocyte are drawn into a 25-28 μm ID enucleation pipette. Enucleation is assessed by visualization of metaphase plate and polar body in the enucleation pipette under UV light and Chroma Technology Hoechst filter set (exciter D360, emitter D460; Hoechst, Strasbourg, Germany). The same enucleation pipette is used to aspirate the dis-aggregated donor cell and place it into the perivitelline space of the enucleated oocyte.

Fusion of NT couples is induced by one 15 μsecond, 2.0 kV/cm DC pulse in a 3.5 mm fusion chamber (BTX, San Diego, Calif.). Fusion medium is 0.25 M D-sorbitol containing 0.5 mM Hepes and 1 mg/ml fatty acid-free BSA. Fused NT embryos are IP3-DMAP-activated and then cultured to the blastocyst stage.

Activation of MII Oocytes

After a 4-hour period following cell fusion, NT embryos are activated by being placed into the 3.5 mm fusion chamber containing 25 μM D-myo-inositol 1,4,5-trisphosphate, hexapotassium salt (IP3, Molecular Probes, Eugene, Oreg.) in Ca²⁺ and Mg²⁺ free PBS plus 100 mM EGTA. Following a brief equilibration period, NT embryos receive two 15 μsecond DC pulses spaced 1 second apart (1.4 kV/cm). The IP3 treated NT embryos are then incubated with 0.2 mM DMAP for 4 hours before being placed into culture medium. The NT embryos are cultured in CR2-complete medium at 39° C. in 5% CO₂ and air for 8 days (day 0=fusion).

This disclosure provides methods for selecting animals (e.g., livestock), particularly for selecting animals for cloning, as well as specific cloning procedures useful in this selection process. It will be apparent that the precise details of these methods and procedures, and the media herewith, may be varied or modified without departing from the spirit of the described embodiments. We claim all such modifications and variations that fall within the scope and spirit of the claims below.

We claim:

1. A method for selecting an animal to be cloned, comprising:
identifying a group of animals;
taking a cell sample from at least one animal;
preserving the cell sample;
obtaining a measurement of at least one characteristic of each animal; and
selecting an animal to be cloned, based on the measurement of the at least one characteristic.

2. The method of claim 1 where at least one characteristic is a post-mortem characteristic.

3. The method of claim 1 where at least one characteristic is an ante-mortem characteristic.

4. The method of claim 1 where preserving the cell sample comprises culturing the cell sample to produce a cell culture.

5. The method of claim 1, comprising obtaining a measurement of more than one characteristic.

6. The method of claim 5 where at least one ante-mortem and at least one post-mortem trait measurement are obtained.

7. The method of claim 5 where at least one characteristic for which a measurement is obtained is an expected progeny difference (EPD) or a governmental quality grade.

8. The method of claim 7 where at least one characteristic is marbling score.

9. The method of claim 5 where at least one characteristic is individual average daily gain.

10. The method of claim 7 where at least average daily gain and marbling score are measured.

11. The method of claim 1 where the animals include cattle, pigs, horses, goats, sheep, chickens, turkeys, mice, rats, monkeys, cats, dogs, reptiles, or captive wild animals.

12. The method of claim 1 where the animals are ruminants.

13. The method of claim 12 where the ruminants are cattle.

14. The method of claim 13 where the cattle comprise hybrid cattle.

15. The method of claim 1 where the cell sample is taken from the animals while the animals are alive.

16. The method of claim 1 where the cell sample is taken from the animals while the animals are dead.

17. The method of claim 1 where the cell sample comprises a fibroblast.

18. The method of claim 1 where preserving the cell sample comprises freezing at least a portion of the cell sample.

19. The method of claim 1 where the preserving the cell sample comprises growing the cell sample in culture to produce an in vitro cell culture.

20. The method of claim 19 where preserving the cell sample comprises freezing at least a portion of the in vitro cell culture.

21. The method of claim 1, further comprising selecting more than one animal to be cloned.

22. The method of claim 21 where the animal selected is a steer.

23. The method of claim 22, further comprising:
cloning the steer to produce a genetically essentially identical bull.

24. A bull as created using the method of claim 23.

25. The method of claim 23, further comprising:
collecting semen from the bull.

26. Semen of claim 25.

27. The method of claim 21 where the animal selected is a reproductively compromised female bovine.

28. The method of claim 27, further comprising:
cloning the female bovine to produce a reproductively competent, genetically essentially identical female bovine.

29. A reproductively competent female bovine of claim 28.

30. The method of claim 28, further comprising:
collecting an egg, an embryo, or a fetus from the reproductively competent, genetically essentially identical female bovine.

31. An egg, an embryo, or a fetus of claim 30.

32. A method of selecting and cloning an animal, comprising:
selecting an animal using the method of claim 1; and
cloning the selected animal from the preserved cell.

33. A method of cloning an animal, comprising:
assembling a group of individual animals;
preserving a sample from at least one individual animal, where the sample contains at least one cell;
obtaining a measurement of at least one post-mortem characteristic of the individual animals;
selecting an animal to be cloned, based on the measurement of the at least one post-mortem characteristic; and
cloning the selected animal from the preserved cell.

34. The method of claim 33, further comprising:
obtaining a measurement of at least one ante-mortem trait; and
selecting an animal to be cloned based on the measurement of both the at least one ante-mortem trait and the at least one post-mortem trait from the same animal.

35. The method of claim 33 where cloning comprises quiescent cell nuclear transfer, proliferating cell nuclear transfer, two-step nuclear transfer cloning, or gonadal cell cloning.

36. The method of claim 33 where cloning the animal comprises:
transferring nuclear material of the preserved cell to a first enucleated oocyte to generate a first couplet;
culturing the first couplet in vitro and/or in vivo for a sufficient length of time and under appropriate conditions to produce a first clone;
transferring nuclear material of a cell of the first clone to a second enucleated oocyte to generate a second couplet; and
generating a cloned animal from the second couplet.

37. The method of claim 36 where the first clone is a fetus when nuclear material is transferred to generate the second couplet.

38. The method of claim 36 where the cell of the fetus is a non-proliferating cell.

39. The method of claim 36 where the preserved cell is a fibroblast.

40. The method of claim 36 where the cell of the fetus is a fibroblast.

41. The method of claim 36 where maturing the first couplet comprises maturing the couplet in vivo to a relative gestational age of at least 30 days.

42. The method of claim 32 where the animal is a bovine animal.

43. The method of claim 33 where the post-mortem characteristic is an expected progeny difference (EPD) or a governmental quality grade.

44. A cloned animal produced by the method of claim 32.

45. A method of selecting and cloning an animal, comprising:
identifying a group of bovine animals;
preserving a sample from each bovine animal, where the sample contains a fibroblast cell;
obtaining a measurement of at least one ante-mortem characteristic of the bovine animals;
slaughtering the group of bovine animals;
obtaining a measurement of at least one post-mortem characteristic of the bovine animals
selecting at least one bovine animal to be cloned, based on at least one ante-mortem and at least one post-mortem measured characteristic; and
cloning the selected bovine animal from the preserved fibroblast cell, where cloning the fibroblast cell comprises:
transferring nuclear material of the preserved fibroblast cell to a first enucleated oocyte to generate a first couplet;
maturing the first couplet in vitro and/or in vivo for a sufficient length of time and under appropriate conditions to produce a fetus of at least 30 days gestational age;
aborting the fetus;
transferring nuclear material of a fibroblast cell of the fetus to a second enucleated oocyte to generate a second couplet; and
generating a cloned animal from the second couplet.

46. A cloned animal produced by the method of claim 45.
